(19) United States
(12) Patent Application Publication
(10) Pub. No.: US 2025/0057774 A1
(43) Pub. Date: Feb. 20, 2025

(54) FAST DISSOLVING SOFTGEL CAPSULES

(71) Applicant: R.P. SCHERER TECHNOLOGIES, LLC, Carson City, NV (US)

(72) Inventors: Soo Ah JIN, St. Petersburg, FL (US); Qi FANG, St. Petersburg, FL (US); Karunakar SUKURU, St. Petersburg, FL (US); Aris GENNADIOS, St. Petersburg, FL (US)

(21) Appl. No.: 18/940,147

(22) Filed: Nov. 7, 2024

Related U.S. Application Data
(63) Continuation of application No. PCT/US23/30042, filed on Aug. 11, 2023.
(60) Provisional application No. 63/397,554, filed on Aug. 12, 2022.

(51) Int. Cl. A61K 9/48; A61K 47/36

(52) U.S. Cl. CPC A61K 9/4816; A61K 47/36

(57) ABSTRACT

Disclosed in certain embodiments is a softgel capsule film comprising a non-gelatin biobased polymer, the film completely dissolving in less than 30 minutes when subject to a dissolution with a USP Apparatus II with paddles at 75 RPM in 900 ml of 0.1N HCL and deionized water at 37 degrees C.

### CROSS REFERENCE TO RELATED APPLICATION(S)

The present application claims priority to U.S. Provisional Patent Application No. 63/397,554 filed on Aug. 12, 2022, the entire contents of which are incorporated in their entirety.

### FIELD OF THE INVENTION

The present disclosure relates to a softgel capsule including a softgel capsule film. The softgel capsule film includes a non-gelatin biobased polymer. The softgel capsule also includes a fill material containing an active agent.

### BACKGROUND

Softgel capsules are ubiquitously utilized in both pharmaceutical and nutraceutical industries. Gelatin is the most abundantly used ingredient for a softgel shell due to its advantages including high aqueous solubility, flexibility and mechanical robustness. Today, there is a high demand in gelatin, causing a deficit supply to meet the growing demand. To mitigate such dependence on gelatin, plant-based softgel capsules have been developed using carrageenan (seaweed extract) in combination with starch.

In order for a compound to serve as a shell material in softgel applications, it must satisfy two important criteria: (1) shell must be robust enough to hold the fill and shape, physically stable and (2) shell needs to be soluble in aqueous medium (physiological media) to release the active pharmaceutical ingredient upon consumption. For an immediate release pharmaceutical dosage form, the expectation is to have at least 80% of the contents release within 30 minutes.

It has been found that such immediate drug release is challenging to achieve with starch and carrageenan because of their poor aqueous solubility. One downside to this approach is that starch has poor water solubility, thereby limiting its use in an immediate release dosage form.

Accordingly, there is a need for improved non-gelatin softgel capsule formulations that have higher aqueous solubility that meet or exceeds immediate release dosage form dissolution requirements and are amenable to high-speed manufacturing as a reliable and commercially viable alternate to gelatin based softgel capsules.

### BRIEF SUMMARY

According to various embodiments, disclosed herein is a softgel capsule film comprising non-gelatin biobased polymer. In certain embodiments, the film completely dissolves in less than 20 minutes when subject to a dissolution with a USP Apparatus II with paddles at 75 RPM in 900 ml of 0.1N HCL and deionized water at 37 degrees C.

According to further embodiments, disclosed herein is a softgel capsule comprising a fill material comprising an active agent that is encapsulated by a film comprising non-gelatin biobased polymer and methods of manufacture and treatment thereof.

### BRIEF DESCRIPTION OF DRAWINGS

FIG. 1 is a representative acetaminophen release profile in dissolution medium with 2% SDS and corresponding photographs at each stage according to Example 1;

FIG. 2 is a representative liquid acetaminophen release profile of 22MC-01 and gelatin capsules according to Example 1;

FIG. 3 is a representative ibuprofen release profiles of 22MC-40 capsules according to Example 1;

FIG. 4 is a representative ibuprofen release profiles of 22MC-02, 22MC-46 and gelatin capsules according to Example 2;

FIG. 5 is a representative ibuprofen release profile of 21MC-96, 22MC-41 and gelatin capsules according to Example 3;

FIGS. 6A-6C is a compiled data set of physical properties and dissolution performance of 22MC-01 after T12M stability study;

FIGS. 7A-7F is a compiled data set of physical properties and dissolution performance of 22MC-02, 22MC-46 after T12M stability study; and

FIGS. 8A-8F is a compiled data set of physical properties and dissolution performance of 21MC-96 and 22MC-41 after T6M and T12M stability study.

### DETAILED DESCRIPTION

Described herein are various embodiments of softgel capsule films and formulations and methods of preparation and use thereof. It is to be understood that the invention is not limited to the details of construction or process steps set forth in the following description. The invention is capable of other embodiments and of being practiced or being carried out in a variety of ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a softgel capsule” includes a single softgel capsule as well as two or more softgel capsules.

As used herein, “free or substantially free,” refers to a composition that comprises less than about 1 wt %, less than about 0.5 wt %, less than about 0.25 wt %, less than about 0.1 wt %, less than about 0.05 wt %, less than about 0.01 wt %, or 0 wt % of said component.

As used herein, “about” refers to any values that are within a variation of ±10%, such that “about 10” would include from 9 to 11. As used herein, “a,” “an,” or “the” refers to one or more, unless otherwise specified. Thus, for example, reference to “an excipient” includes a single excipient as well as a mixture of two or more different excipients, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

As used herein, the term “film,” “film composition,” “shell” or “shell composition” refers to the shell of a softgel capsule which encapsulates a fill material.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that are within the scope of the appended claims and their equivalents.

Disclosed herein is a softgel capsule film comprising a non-gelatin biobased polymer. In certain embodiments, the film may completely dissolve in less than 30 minutes when subject to a dissolution with a USP Apparatus II with paddles at 75 RPM in 900 ml of 0.1N HCL and deionized water at 37 degrees. The softgel capsule film may dissolve in less than 25 minutes, less than 20 minutes, less than 15 minutes, less than 10 minutes, or less than 5 minutes.

In certain embodiments of the softgel capsule film, the non-gelatin biobased polymer includes stearic acid, maltodextrin, pullulan, or a combination thereof.

In certain embodiments of the softgel capsule film, the non-gelatin biobased polymer may be included in an amount of about 1% to about 25% (w/w), about 3% to about 22% (w/w), about 5% to about 20% (w/w), about 7.5% to about 17.5% (w/w), or about 10% to about 15% (w/w), based on total weight of the film, or any range, sub-range or value within.

In certain embodiments of the film, the non-gelatin biobased polymer may be pullulan. In other embodiments of the film, the non-gelatin biobased polymer may be stearic acid. In other embodiments of the film, the non-gelatin biobased polymer may be maltodextrin.

In certain embodiments of the film, pullulan may be in the film in an amount of about 1% to about 20%, about 2% to about 18%, about 4% to about 16%, about 5% to about 15%, or about 7.5% to about 12.5% (w/w) of the film, or any range, sub-range or value within.

In some embodiments of the film, stearic acid may be in the film in an amount of about 0.5% to about 5%, about 1% to about 4%, or about 2% to about 3% (w/w) of the film, or any range, sub-range or value within.

In certain embodiments of the film, the non-gelatin biobased polymer may be pullulan and stearic acid in an amount of about 1.5% to about 25%, about 2% to about 20%, about 3% to about 18%, about 5% to about 15%, or about 7.5% to about 12. % (w/w) of the film, or any range, sub-range or value within.

In certain embodiments, the film may further include a synthetic polymer. The synthetic polymer may include polyvinyl alcohol, polyvinyl alcohol-polyethylene glycol graft co-polymer, high molecular weight polyethylene glycol, povidone, a surfactant, a nonionic triblock copolymer or a combination thereof. In some embodiments, the synthetic polymer may be a nonionic triblock copolymer. The nonionic triblock copolymer may include polyethylene oxide and polypropylene oxide blocks. In some embodiments, the surfactant may include sodium lauryl sulfate. In some embodiments of the film, the synthetic polymer may be in the film in an amount of about 4% to about 8%, or about 5% (w/w) of the film.

In certain embodiments, the film may further include a non-animal derived gelling agent. The non-animal derived gelling agent may include carrageenan, starch, pregelatinized starch, xanthan gum, agar, pectin, sugar, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, egg white powder, lacto albumin, soy protein, chitosan or a combination thereof.

In certain embodiments, the non-animal derived gelling agent may include carrageenan, starch, or a combination thereof. In some embodiments, the carrageenan may be iota carrageenan, kappa carrageenan, lambda carrageenan, or a combination thereof.

In some embodiments, the starch may include modified starch, potato starch, corn starch, tapioca starch, hydroxy propylated starch, hydroxyalkylated starch, acid-treated starch, dextrin or a combination thereof.

In certain embodiments, the ratio of carrageenan to starch may be about 1:1 to about 1:10, about 1:1 to about 1:8, about 1:1 to about 1:5, or about 1:2.5 to about 1:4.5.

In certain embodiments, the non-animal gelling agent does not include a starch.

In certain embodiments, the non-animal gelling agent may be in an amount of from about 15% to about 60% (w/w) of the film. In some embodiments, the non-animal gelling agent may be included in an amount of from about 20% to about 55%, about 25% to about 50%, about 30% to about 45%, or about 35% to about 55% (w/w) of the film. In some embodiments, the film may include carrageenan in an amount of about 5% to about 20%, about 8% to about 18%, or about 10% to about 15% (w/w) of the film. In some embodiments, the film may include starch in an amount of about 0 to about 45%, about 5% to about 40%, about 10% to about 35%, about 15% to about 30%, or about 20% to about 25% (w/w) of the film.

In other embodiments, the film may include less than 10%, less than 5% or less than 1% (w/w) of an animal derived gelling agent based. In certain embodiments, the softgel capsule film does not contain an animal derived gelling agent.

In certain embodiments, the softgel capsule film may also include a plasticizer. In other embodiments, the film may also include a buffer agent.

In some embodiments, the plasticizer may be glycerol, glycerin, sorbitol, sorbitol sorbitan solution, triacetin, polysorbate or combinations thereof. In some embodiments, the polysorbate may include polysorbate 20 also known as Tween 20, polysorbate 80 also known as Tween 80, or a combination thereof. In certain embodiments of the film, the plasticizer may be included in an amount of about 15% to about 40%, about 20% to about 35%, or about 25% to about 30% (w/w) of the film.

In some embodiments, the buffer agent may be dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate, dibasic potassium phosphate or a combination thereof. In some embodiments of the film, the buffer agent may be in an amount of about 0.1% to about 5% (w/w) of the film. In other embodiments, the buffer agent may be included in an amount of about 0.1% to about 5%, about 0.3% to about 4.5%, about 0.5% to about 4%, about 1% to about 3.5%, or about 1.5% to about 3%, or any value or sub-range herein.

In certain embodiments, the film includes an amount of gelatin, e.g., less than about 50% w/w of the film, less than about 40% w/w of the film, less than about 25% w/w of the film, less than about 10% w/w of the film, less than about 5% w/w of the film, less than about 3% w/w of the film or less than about 1% w/w of the film.

Also disclosed in certain embodiments is a softgel capsule formulation comprising a fill material comprising an active agent, wherein the fill material is encapsulated by a film composition as disclosed herein.

In certain embodiments, a softgel capsule includes a film composition including a non-gelatin biobased polymer. The non-gelatin biobased polymer may include maltodextrin, pullulan, carrageenan, or a combination thereof.

In certain embodiments of the softgel capsule, the film composition may further include a synthetic polymer. The synthetic polymer may be polyvinyl alcohol, polyvinyl alcohol-polyethylene glycol graft co-polymer, high molecular weight polyethylene glycol, povidone, a surfactant, a nonionic triblock copolymer, or a combination thereof. In some embodiments, the synthetic polymer may be a nonionic triblock copolymer. In some embodiments, the nonionic triblock copolymer may include polyethylene oxide and polypropylene oxide blocks. In some embodiments, the surfactant may be sodium lauryl sulfate.

In certain embodiments of the softgel capsule, the film composition may further include a non-animal derived gelling agent. The non-animal derived gelling agent may include carrageenan, starch, xanthan gum, agar, pectin, sugar, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, egg white powder, lacto albumin, soy protein, chitosan or a combination thereof.

In certain embodiments of the softgel capsule, the film may further include a plasticizer. The plasticizer may be glycerol, glycerin, sorbitol, sorbitol sorbitan solution, triacetin, polysorbate, or combinations thereof. In some embodiments, the polysorbate includes Tween 20, Tween 80 or a combination thereof. In some embodiments, the plasticizer may be included in an amount of about 15% to about 40% (w/w) based on the film. In some embodiments, the plasticizer may be sorbitol sorbitan solution.

In certain embodiments of the softgel capsule, the film may further include a buffer agent. The buffer agent may be dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate, dibasic potassium phosphate and a combination thereof.

In certain embodiments of the softgel capsule, the film does not include an animal derived gelling agent.

Also disclosed herein is an immediate release softgel capsule including a fill material encapsulated by a film composition, wherein the film composition includes a non-gelatin biobased polymer, wherein the film dissolves in less than about 20 minutes according to a dissolution test using USP Apparatus paddle II at 75 rpm. In certain embodiments, the fill material may include an active agent.

In certain embodiments, an immediate release softgel capsule includes a fill material encapsulated by a film composition, wherein at least 80% of the fill material releases within 30 minutes.

The film composition as disclosed herein may also contain at least one of a buffering agent, a plasticizer and water. Softshell capsule formulations as described herein can be vegetarian and free of animal derived materials such as gelatin. The immediate release softgel capsule may include a film composition as described above in the present disclosure.

In certain embodiments, the film disclosed herein completely dissolves in less than 25 minutes, less than 20 minutes, less than 15 minutes, less than 10 minutes, or less than 5 minutes when subject to a dissolution with a USP Apparatus II with paddles at 75 RPM in 900 ml of 0.1N HCL and deionized water at 37 degrees C.

In certain embodiments, the softgel capsule formulation contains water. The water may be present in an amount of about 30 wt % to about 60 wt %, or about 35 wt % to about 55 wt %, or about 40 wt % to about 50 wt %, or about 42 wt %, or about 43 wt %, or about 44 wt %, or about 45 wt %, or about 45.5 wt %, or about 46 wt %, or about 47 wt %, or about 48 wt % of the film.

In an embodiment, the shell composition/film of the softgel capsule may optionally comprise additional agents such as coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

The softshell capsule formulations as disclosed herein may further include a fill composition. The fill composition may contain at least one of rapeseed oil, medium chain triglyceride oil, polyethylene glycol and combinations thereof. Lipophilic and/or hydrophilic and/or alcohol fill compositions could also be encapsulated with the softshell capsule formulations as described herein.

Any pharmaceutically active ingredient may be used for purposes of the present disclosure, including both those that are water-soluble and those that are poorly soluble in water. Suitable pharmaceutically active ingredients include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof. In certain embodiments, the present invention is also directed to methods of treatment utilizing any of the active ingredients disclosed herein to treat a disease or condition that can be treated by the active ingredient.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In some embodiments, the lipids in the dosage form may be selected, without limitations, from the group consisting of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, animal or vegetable fats, e.g., in their hydrogenated form, free fatty acids and mono-, di-, and tri-glycerides with C8-, C10-, C12-, C14-, C16-, C18-, C20- and C22-fatty acids, and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lornoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin, pharmaceutically acceptable salts thereof and mixtures thereof, acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, methylphenidate as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2, 4, 6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

Suitable fill materials comprise at least one active ingredient and can be made according to known methods. In addition to the at least one active ingredient, suitable fill materials may comprise additional fill components such as flavoring agents, sweetening agents, coloring agents and fillers or other pharmaceutically acceptable excipients or additives such as synthetic dyes and mineral oxides. Suitable amounts of pharmaceutically active ingredient and pharmaceutically acceptable excipients can be readily determined by one of ordinary skill in the art.

The disintegration tests disclosed herein were performed at about 37° C.±2° C. at a volume of fluid of 1000 mL. Disintegration test fluid 1 (also referred to herein as “artificial gastric juice”) was 2 g/L sodium chloride-hydrochloric acid solution having a pH of 1.2. Disintegration test fluid 2 (also referred to herein as “artificial intestinal fluid”) was 0.2 mol/L potassium dihydrogen phosphate—0.2 mol/L sodium hydroxide solution having a pH of 6.8.

The disintegration test with the first fluid was carried out for about 120 minutes by placing one unit in each of the six tubes of the basket, immersing the basket (and consequently the units) in the first test fluid, and lifting the basket from the fluid to observe whether the units disintegrated. Disintegration is defined as that state at which the unit is broken or the enteric shell composition is ruptured or broken. The test is met if none of the six units is disintegrated. A similar test is performed with the second disintegration test fluid for the selected duration.

In some embodiments, the disintegration test may be performed for about 150 minutes, about 120 minutes, about 105 minutes, about 90 minutes, about 75 minutes, about 60 minutes, about 45 minutes, about 30 minutes, about 15 minutes, about 10 minutes, or about 5 minutes.

In some embodiments, the softgel capsule may have a burst strength of about 6 kg to about 20 kg, 8 kg to about 15 kg, about 10 kg to about 15 kg, about 8 kg, about 10 kg, about 12 kg, or about 15 kg at 1 month, 3 months, 6 months or 12 months at 40° C./75% RH.

In some embodiments, the softgel capsule may have a burst strength of about 6 kg to about 20 kg, 8 kg to about 15 kg, about 10 kg to about 15 kg, about 8 kg, about 10 kg, about 12 kg, or about 15 kg at 1 month, 3 months, 6 months or 12 months at 30° C./65% RH.

In some embodiments, the softgel capsule may release 80% of the fill material after about 30 minutes, about 35 minutes, about 40 minutes, about 45 minutes, about 50 minutes, or about 55 minutes, e.g, in a dissolution test in 900 mL phosphate buffer (50 mM, pH 7.2), e.g., at 37 degrees, using USP APP II with paddle at 75 RPM or dissolution with a USP Apparatus II with paddles at 75 RPM in 900 ml of 0.1N HCL and deionized water, e.g., at 37 degrees. In certain embodiments, the above results may also be achieved for a softgel capsule under accelerated stability conditions of 25° C./60% RH, 30° C./65% RH, or 40° C./75% RH at 1 month, 3 months, 6 months, 12 months, 18 months, 22 months, or 24 months. In certain embodiments, after any of the accelerated storage conditions disclosed herein, the dissolution does not change from time 0 by more than 20%, by more than 10% or by more than 5% at 1 hour, 4 hours or 8 hours.

In some embodiments of the softgel capsule, the softgel capsule may have a water activity (Aw) may be about 0.3 to about 1.0, about 0.4 to about 0.9, about 0.5 to about 0.8, about 0.3, about 0.4, about 0.5, about 0.6, about 0.7, about 0.8, about 0.9, or about 1.0 at 1 month, 3 months, 6 months, or 12 months at 40° C./75% RH.

In some embodiments of the softgel capsule, the softgel capsule may have a water activity (Aw) may be about 0.3 to about 1.0, about 0.4 to about 0.9, about 0.5 to about 0.8, about 0.3, about 0.4, about 0.5, about 0.6, about 0.7, about 0.8, about 0.9, or about 1.0 at 1 month, 3 months, 6 months, or 12 months at 30° C./65% RH.

### Methods of Preparing the Dosage Forms

Disclosed herein are methods of preparing a softgel capsule film and capsule formulation as disclosed herein. The methods include combining a non-gelatin biobased polymer and optionally at least one of a synthetic polymer, non-animal derived gelling agent, buffering agent, a plasticizer and water to form a combination.

The method may also further include transferring the combination to an encapsulation apparatus. In certain embodiments, the method may include encapsulating a fill material within a softgel capsule formed from the combination to form a plurality softgel capsule dosage forms. The method may also further include drying the plurality of softgel capsule dosage forms in a tumble dryer. Certain embodiments further include packaging the plurality of softgel capsule dosage forms.

In certain embodiments, the present invention is directed to the one or more of the following lists of items:
1. A softgel capsule film including:
   a non-gelatin biobased polymer, wherein the film dissolves in less than 20 minutes according to a dissolution test using USP Apparatus paddle II at 75 rpm.
2. The softgel capsule film of item 1, wherein the non-gelatin biobased polymer comprises stearic acid, maltodextrin, pullulan, or a combination thereof.
3. The softgel capsule film of items 1 or 2, further comprising a synthetic polymer.
4. The softgel capsule of item 3, wherein the synthetic polymer is polyvinyl alcohol, polyvinyl alcohol-polyethylene glycol graft co-polymer, high molecular weight polyethylene glycol, povidone, a surfactant, a nonionic triblock copolymer or a combination thereof.
5. The softgel capsule of items 3 or 4, wherein the synthetic polymer is a nonionic triblock copolymer.
6. The softgel capsule of any one of items 3-5, wherein the nonionic triblock copolymer comprises polyethylene oxide and polypropylene oxide blocks.
7. The softgel capsule film of items 3 or 4, wherein the surfactant is sodium lauryl sulfate.
8. The softgel capsule film any one of the preceding claims, further comprising a non-animal derived gelling agent.
9. The softgel capsule film of item 8, wherein the non-animal derived gelling agent comprises carrageenan, starch, pregelatinized starch, xanthan gum, agar, pectin, sugar, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, egg white powder, lacto albumin, soy protein, chitosan or a combination thereof.
10. The softgel capsule film of any one of the preceding items, further comprising a plasticizer.
11. The softgel capsule film of any one of the preceding items, further comprising a buffer agent.
12. The softgel capsule film of any one of the preceding items, wherein the film dissolves in less than 15 minutes, less than 10 minutes, or less than 5 minutes.
13. The softgel capsule film of any one of the preceding items, wherein the non-animal derived gelling agent comprises carrageenan, starch, or a combination thereof.
14. The softgel capsule film of any one of the preceding items, wherein the ratio of carrageenan to starch is about 1:1 to about 1:10, about 1:1 to about 1:8, about 1:1 to about 1:5 or about 1:2.5 to about 1:4.5.
15. The softgel capsule film of item 13, wherein the carrageenan comprises iota carrageenan, kappa carrageenan, lambda carrageenan or a combination thereof.
16. The softgel capsule film of item 13, wherein the starch comprises modified starch, potato starch, corn starch, tapioca starch, hydroxy propylated starch, hydroxyalkylated starch, acid-treated starch, dextrin and a combination thereof.
17. The softgel capsule film of item 8, wherein the non-animal gelling agent does not include a starch.
18. The softgel capsule film of item 8, wherein the non-animal gelling agent is in an amount of from about 15% to about 60% (w/w) of the film.
19. The softgel capsule film of item 13, wherein the carrageenan is in an amount of about 5 to about 20% (w/w).
20. The softgel capsule film of item 13, wherein the starch is in an amount of about 0 to about 45% (w/w).
21. The softgel capsule film of any of the preceding items, comprising less than 10%, less than 5%, or less than 1% of an animal derived gelling agent.
22. The softgel capsule film of any of the preceding items, wherein the film does not contain an animal derived gelling agent.
23. The softgel capsule film of item 10, wherein the plasticizer comprises glycerol, glycerin, sorbitol, sorbitol sorbitan solution, triacetin, polysorbate or combinations thereof.
24. The softgel capsule film of item 23, wherein the polysorbate comprises Tween 20, Tween 80 or a combination thereof.
25. The softgel capsule film of item 10, wherein the plasticizer is in an amount of from about 15 to about 40% (w/w) of the film.
26. The softgel capsule film of item 11, wherein the buffer agent is in an amount of from about 0.1 to about 5% (w/w) of the film.
27. The softgel capsule film of item 11, wherein the buffer agent is selected from dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate, dibasic potassium phosphate and a combination thereof.
28. The softgel capsule film of any of the preceding items, wherein the polymer is in an amount of from about 1 to about 25% (w/w), about 3 to about 22% (w/w), about 5 to about 20% (w/w), about 7.5 to about 17.5% (w/w), or about 10 to about 15% (w/w) of the film.
29. The softgel capsule film of item 2, wherein the polymer is pullulan.
30. The softgel capsule film of item 29, wherein pullulan is in an amount of about 1 to about 20% (w/w), about 2 to about 18% (w/w), about 4 to about 16% (w/w), about 5 to about 15% (w/w), or about 7.5 to about 12.5% (w/w) of the film.
31. The softgel capsule film of item 2, wherein the polymer is stearic acid.
32. The softgel capsule film of item 31, wherein stearic acid is in an amount of about 0.5 to about 5% (w/w), about 1 to about 4% (w/w), or about 2 to about 3% (w/w) of the film.
33. The softgel capsule film of item 2, wherein the polymer is pullulan and stearic acid.
34. The softgel capsule film of item 33, wherein the polymer is in an amount of about 1.5 to about 25% (w/w), about 2 to about 20% (w/w), about 3 to about 18% (w/w), about 5 to about 15% (w/w), or about 7.5 to about 12.5% (w/w) of the film.
35. The softgel capsule of item 3, wherein the synthetic polymer is in an amount of about 4 to about 8% (w/w), or about 5% (w/w) of the film.
36. An immediate release softgel capsule comprising a fill material encapsulated by a film composition, wherein the film composition comprises a non-gelatin biobased polymer, wherein the film dissolves in less than 20 minutes according to a dissolution test using USP Apparatus paddle II at 75 rpm.
37. The immediate release softgel capsule of item 36, wherein the fill material comprises an active agent.
38. An immediate release softgel capsule comprising a fill material encapsulated by a film composition, wherein the film composition comprises a non-gelatin biobased polymer, wherein at least 80% of the fill material release within 30 minutes.
39. The immediate release softgel capsule of item 38, wherein the fill material comprises an active agent.

### EXAMPLES

### Example 1—Partial Replacement of Starch with Kolliphor P407 (Poloxamer)

Starch was partially replaced with Kolliphor P407, nonionic triblock copolymer consisting of hydrophilic (polyethylene oxide, PEO) and hydrophobic (polypropylene oxide, PPO) blocks. Kolliphor P407 was selected due to its excellent aqueous solubility and thermo-reversible gelling behavior. Softgels were manufactured using the gel mass as shown in Table 1.

TABLE 1
Gel Mass Formula Containing Kolliphor P407

| | | % (based on |
|---|---|---|
| Item Number | Item Description | weight of film) |
| 00308270 | Modified Starch | 10-42 |
| 00308305 | Carrageenan | 5-18 |
| 00308274 | Sodium Phosphate Dibasic Anhydrous | 0.4-2.0 |
| N/A | Polyethylene-polypropylene | 4-8 |
| | glycol (Kolliphor P407 Geismar) | |
| 00308197 | Sorbitol sorbitan solution | 18-32 |
| 00391173 | Pullulan | 1-14 |

Oil-based fill and high molecular PEG-based fill was used for encapsulation. Acetaminophen (325 mg per softgel) was suspended in soybean oil and the suspension was used to encapsulate 900 mg softgel (OET-10291399 Lot #22MC-01). Ibuprofen (200 mg per softgel) was dissolved in a mixture of PEG1000 and PEG600 at 9:1 ratio and the mix was used to encapsulate 360 mg softgel (OET-10291399 Lot #22MC-40).

After the softgel capsules were dried, they were washed and placed in plastic bags prior to testing.

Burst strength measurement and dissolution tests using fiberoptics probes were conducted to evaluate the softgel physical properties and drug release kinetics, respectively. Dissolution setup was prepared as per FDA dissolution methods with slight modification. 2% of sodium dodecyl sulfate (SDS) was added to 900 mL water to release API trapped inside oil globules for 22MC-01 softgel capsules and 900 mL of 50 mM phosphate buffer at pH 7.2 was used as dissolution medium for 22MC-40 softgel capsules. Dissolution medium was agitated using paddle apparatus at 100 RPM unless other specified.

### Example 2—Partial Replacement of Starch with Stearic Acid

Stearic acid is a saturated fatty acid with 18-carbon chain. Stearic acid is known to form lipid-amylose complex during gelatinization and hence concomitantly retards the recrystallization/retrogradation process of starch during cooling. As the aqueous solubility of starch is highly dictated by its recrystallization rate during cooling, starch was partially replaced with stearic acid. Softgels were manufactured using the gel mass as shown in Table 2.

TABLE 2
Gel Mass Formula Containing Stearic Acid

| | | % (based on |
|---|---|---|
| Item Number | Item Description | weight of film) |
| 00308270 | Modified Starch | 15-42 |
| 00308305 | Carrageenan | 5-18 |
| 00391185 | Potassium Phosphate Dibasic Anhydrous | 0.4-2.0 |
| N/A | Stearic Acid | 0.5-5.0 |
| 00308197 | Sorbitol sorbitan solution | 14-36 |
| 00391173 | Pullulan | 1-16 |

Ibuprofen (200 mg per capsule) was encapsulated in 360 mg softgels (OET-10291401 Lot #22MC-02 and Lot #22MC-46). After the softgel capsules were dried, they were washed with ethanol-phosal 53 MCT wash solution and stored in plastic bags prior to testing.

Burst strength measurement and fiberoptics probes were conducted to evaluate the softgel physical properties and drug release kinetics disclosed herein, respectively. Dissolution setup was prepared as per FDA dissolution method for ibuprofen softgel capsules with slight modification. A dissolution test in 900 mL phosphate buffer (50 mM, pH 7.2) using USP APP II with paddle at 75 RPM was conducted.

Informal stability was initiated and collected 1 month (TIM) data at 40° C./75% RH for Lot #22MC-02 capsules.

### Example 3—Complete Replacement of Starch with Maltodextrin and Pullulan

Maltodextrin is hydrolyzed starch and exhibits higher water solubility than hydroxypropylated starch. Pullulan is a naturally occurring polysaccharide produced by yeast. Starch was completely replaced with maltodextrin and pullulan to achieve robust shell with higher shell solubility. Softgels were manufactured using the gel mass as shown in Table 3.

TABLE 3
Gel Mass Formula Containing Maltodextrin and Pullulan

| | | % (based on |
|---|---|---|
| Item Number | Item Description | weight of film) |
| 00391172 | Maltodextrin M100 | 15-42 |
| 00391173 | Pullulan | 1-15 |
| 00308305 | Carrageenan, NF | 5-16 |
| 00391185 | Potassium Phosphate Dibasic Anhydrous | 0.1-2.0 |
| 00308197 | Sorbitol sorbitan solution | 15-36 |

Ibuprofen (200 mg per capsule) was encapsulated in 360 mg softgels (OET-10291370 Lot #21MC-96 and Lot #22MC-41). After the softgel capsules were dried, they were washed with Ethanol-Phosal mixture and placed in plastic bags prior to testing.

### Summary of Results

### Example 1—Partial Replacement of Starch with Kolliphor P407

### Oil-Based Fill

Table 4 summarizes the softgel burst strength and travel distance of 22MC-01 capsules. Dried 22MC-01 softgel capsules showed moderate burst strength of 15 kg.

TABLE 4
Softgel Bust Strength and Travel Distance
of Dried 22MC-01 Capsules

| Parameter | Data |
|---|---|
| Burst Strength (kg) | 14.8 ± 1.60 |
| Travel Distance (mm) | 3.61 ± 0.09 |

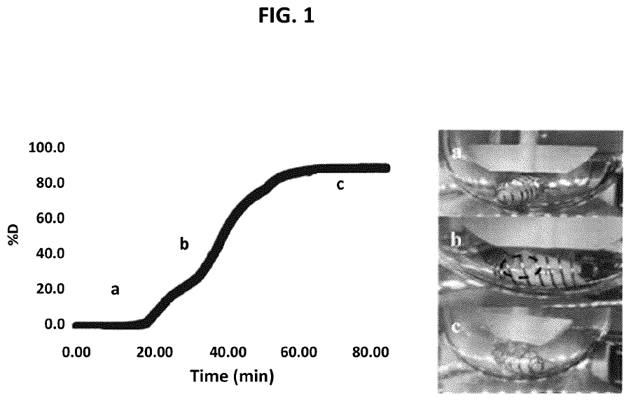

Initial fill release occurred after 14±2 minutes of exposure as the tip of the capsule opened. Shell dissolved simultaneously as the fill released into the dissolution medium. However, the drug dissolved slowly as represented by FIG. 1 regardless of the shell being fully opened and exposed to the surrounding. As shown in the photograph in FIG. 1, the drug was trapped inside coagulated oil globules and thereby delayed the release into the dissolution medium. Despite having the majority of white coagulated oil globules released into the dissolution medium, it took average 56±8 minutes to reach 80% of drug release. This indicated that the delayed release was not due to shell solubility but rather on drug solubility and fill formulation.

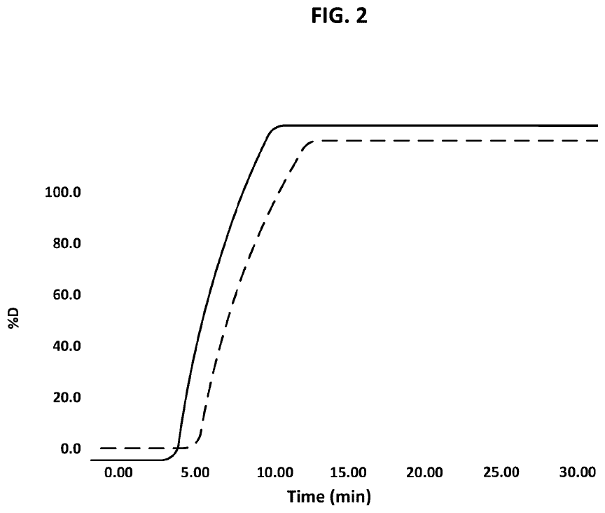

To eliminate the impact of fill formulation on drug solubility and solely evaluate shell solubility, 22MC-01 capsules were filled with liquid acetaminophen and corresponding release profile was measured. Commercially available liquid acetaminophen gelatin softgel capsules were tested in parallel to compare 22MC-01 with conventional gelatin-based capsules. Resulting release profiles are shown in FIG. 2.

When decoupled the drug solubility effect, 22MC-01 release profile matched that of commercial gelatin-based capsules.

Informal stability is ongoing, and corresponding 1 month (T1 M) data is collected at 40° C./75% RH.

### High Molecular PEG-Based Fill

Previous work showed poor compatibility between PEG and the shell material, as all the capsules leaked during drying. It was observed that shell was susceptible to PEG migration and water in the shell facilitated the transport. Incorporating higher molecular weight PEG1000 was expected to mitigate the migration but was not enough to completely halt the migration upon drying. Approximately 40% of the capsules remained intact without any leakers.

Due to PEG migration, the seal integrity was compromised and the resulting softgel burst strength and travel distance of the capsules are reported in Table 5.

TABLE 5
Softgel Bust Strength and Travel Distance
of Dried 22MC-40 Capsules

| Parameter | Data |
|---|---|
| Burst Strength (kg) | 3.31 ± 1.08 |
| Travel Distance (mm) | 3.03 ± 1.43 |

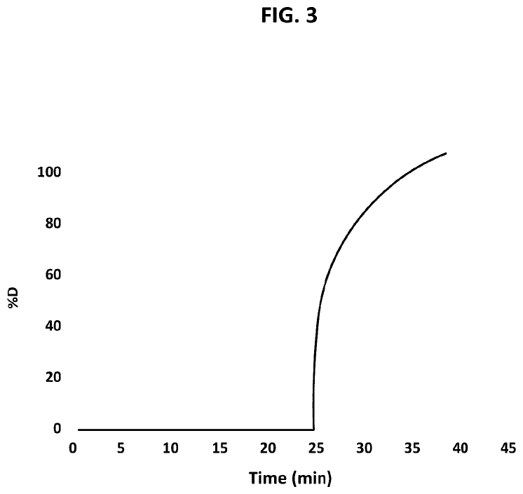

Intact capsules were packaged for further testing and Ibuprofen release profiles were collected using Fiberoptics as shown in FIG. 3.

Unexpectedly, initial fill release occurred after 22±4 minutes of exposure as the tip of the capsule opened. Shell dissolved simultaneously as the fill released into the dissolution medium. Once the capsules split open, the fill released into the dissolution media and 80% of the fill was released at 33±4 minutes. Regardless, the result was better than that of the current commercial capsules, which took 34±16 minutes to initially release the drug and 44±21 minutes to reach 80% fill release.

### Example 2—Partial Replacement of Starch with Stearic Acid

Table 6 summarizes the softgel burst strength and travel distance of capsules from lot 22MC-02 capsules. Dried 22MC-02 softgel capsules showed moderate burst strength of 14 kg.

TABLE 6
Softgel Bust Strength and Travel Distance
of Dried 22MC-02 Capsules

| Parameter | Data |
|---|---|
| Burst Strength (kg) | 14.1 ± 4.2 |
| Travel Distance (mm) | 3.69 ± 0.22 |

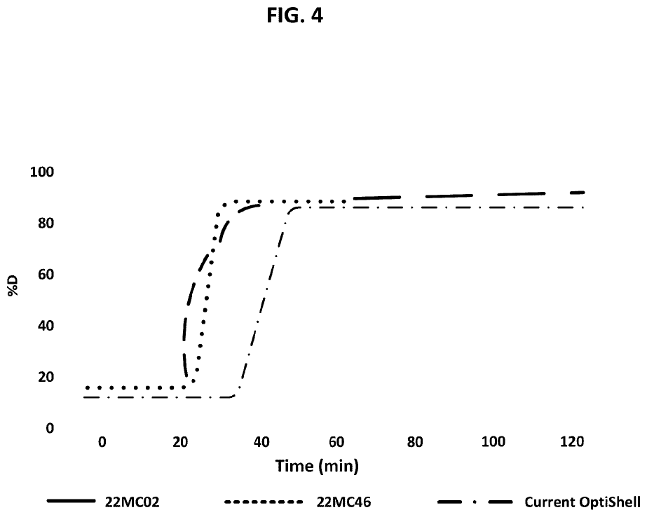

Ibuprofen release profile was collected using Fiberoptic dissolution apparatus using USP App II at 75 rpm and shown in FIG. 4.

FIG. 4 showed the rupture and dissolution profile of capsules from 22MC-02 and 22MC-46 when exposed to dissolution medium. 22MC-02 capsule took 21±6 and 22 MC-46 capsule took 22±1 minutes to initially release the fill. This value was faster than current commercial products, which took 34±16 minutes to initially release the drug. Despite the delay, 80% of the drug released in 35±6 minutes and 33±5 minutes for 22MC-02 and 22MC-46 capsules, respectively. This was faster than regular commercial capsules that took 44±21 minutes to release 80% of the drug.

### Example 3—Partial Replacement of Starch with Stearic Acid

Table 7 summarizes the softgel burst strength and travel distance of 21MC-96 capsules. Softgel burst strength is extremely low of 5 kg; however, such low strength was attributed to the improper die and wedge setup during encapsulation.

TABLE 7
Softgel Bust Strength and Travel Distance
of Dried 21MC-96 Capsules

| Parameter | Data |
|---|---|
| Burst Strength (kg) | 5.94 ± 2.35 |
| Travel Distance (mm) | 4.47 ± 1.30 |

It was found that the capsules had air bubbles trapped inside, which was due to an unsecured wedge that could have compromised the seal integrity as well. A new minicap batch (22MC-41) was produced with no air bubbles.

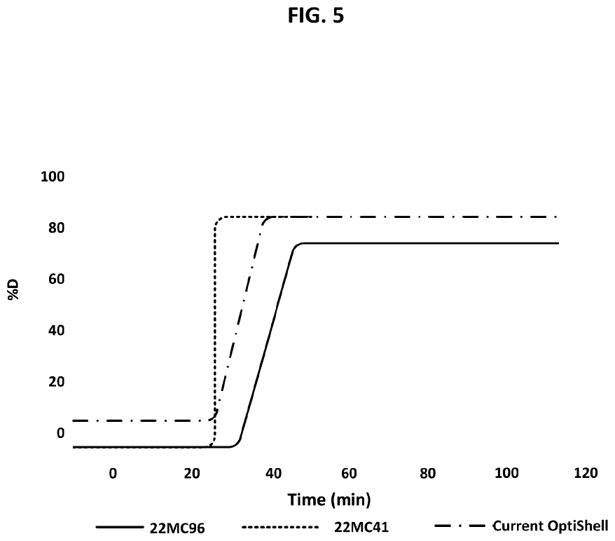

Ibuprofen release profile was collected using Fiberoptic dissolution apparatus at 75 RPM with USP Apparatus II and shown in FIG. 5.

21MC-96 capsules started to dissolve instantaneously when exposed to dissolution medium and initially released the fill after 11±4 minutes. This value was comparable to the gelatin-based softgel capsules, that took less than 10 minutes to initially release the drug. 80% of the fill was released in 28±8 minutes, which was faster than existing non-animal softgels. Unlike starch-based capsules that turned opaque in dissolution medium, starch-free 21MC-96 capsules remained transparent and formed hydrogel throughout the run.

Repeat of 22MC-96 with proper wedge setup (22MC-41) showed delayed fill release compared to 22MC-96, shown in FIG. 5, which was due to better seal quality. Initial fill release occurred after 16±6 minutes and 80% of drug release happened 32±5 minutes. Both values were faster than exiting non-animal softgels.

### Dissolution F₂ Similarity and Mahalanobis Distance Calculation Results

F₂ similarity test was performed to evaluate the difference in drug release profiles among the softgels of the present invention and original non-animal softgels. Amount of drug dissolved (% D) at six different time points (5, 10, 20, 30, 45 and 60 minutes) were collected and f₂ values were calculated using the equation below:

$${f}_{2} = 50 log ( \frac{100}{\sqrt{1 + \frac{\sum {[ R ( t ) - T ( t ) ]}^{2}}{n}}} )$$

R(t)=% D of drug dissolved of reference product at time point t
T(t)=% D dissolved of test product at time point t
n=number of time points

Table 8 summarized the average % D values at each time and f₂ value of each lot

TABLE 8
% D at different time points and corresponding
f₂ values of each newly manufactured softgels

| | Original | 22MC- | 22MC- | 22MC- | 22MC- | 22MC- |
|---|---|---|---|---|---|---|
| Time point | Softgel | 96 | 02 | 40 | 41 | 46 |
| (min) | % D | % D | % D | % D | % D | % D |
| 5 | 0.98 | 3.07 | 1.58 | 1.56 | 4.72 | 1.17 |
| 10 | 0.70 | 4.12 | 1.99 | 1.54 | 5.63 | 1.35 |
| 20 | 1.13 | 55.54 | 5.47 | 6.69 | 9.63 | 1.49 |
| 30 | 1.07 | 83.21 | 45.74 | 61.94 | 60.65 | 59.35 |
| 45 | 41.74 | 91.77 | 94.58 | 95.78 | 98.98 | 97.61 |
| 60 | 87.87 | 96.15 | 98.88 | 99.69 | 100.98 | 99.97 |

| | | | | | |
|---|---|---|---|---|---|
| F₂ | 15.28 | 25.41 | 21.88 | 21.44 | 22.11 |

Based on the f₂ analysis, the inventive softgels showed much faster dissolution compared to the original non-animal softgels and hence the f₂ values are <50 indication the dissolution from the new softgels is not similar to the exiting and is faster and more consistent with less variability.

Mahalanobis distance (MD) is a measure of the distance between sample point P and a distribution D. The distance represents how apart the measurement point is from the distribution mean by specifying in number of standard deviations.

At initial stages (5 and 10 minutes), there was no significant difference in the % release between the control and new softgels, indicating absence of drug in the dissolution medium. The differences appear at 20 minutes and become more pronounced at 30 minutes where the % releases are a few standard deviations away from the average control deviations, which is indicated by the orange color. The difference then becomes less significant after 45 minutes since at that time, control samples begin to rupture and release the drug.

MD analyses further support the faster dissolution of the newly developed softgels compared to original softgels.

Release Profile after 1 Month (T1M) at 40° C./75% RH Storage Condition

An informal stability study was initiated for 22MC-01, 22MC-02, 21MC-96, 22MC-41 and 22MC-46 capsules under 40° C./75% RH conditions. After 1 month, data was collected. No leakers were detected and dissolution profiles were unchanged after 1M accelerated stability study for all three lots that completed 1M stability.

The data supports using a non-gelatin biobased polymer (stearic acid, maltodextrin and pullulan) and/or a synthetic polymer (Kolliphor P407) for fast dissolving non-gelatin oral softgels. Compared to current softgel capsules, all newly developed softgel capsules exhibited relatively superior performance with faster initial drug release within 20 minutes of exposure. For oil-filled encapsulated capsules (22MC-01), it took 55 minutes to reach 80% of drug release; however, such a slow release was attributed to poor drug solubility and fill formulation rather than shell dissolution. This conclusion was further proven through dissolution of pre-dissolved drug solution (using Acetaminophen as a model drug) filled into the air fill capsules made (shell formula of lot 22MC-01). When higher molecular weight PEG was encapsulated (22MC-40), it took longer (22 minutes) to initially release; however, 80% of the fill was released after 33 minutes. Ibuprofen encapsulated capsules (22MC-01 and 21MC-96) showed superior dissolution performance to that of current softgel capsules. Newly developed capsules exhibited more consistent drug release performance compared to the current softgel capsules as evident from the smaller error bar.

Based on this development work, Shell formula used in lot 21MC-96/22MC-41 will be considered for further process development and scale up as they meet the dissolution criteria of not less than 80% drug release within 30 minutes needed for immediate release pharmaceutical dosage forms. Even though stearic acid capsules showed compared dissolution, the capsules are cloudy due to stearic acid and hence won't be considered for clear liquid fill capsules. However, they would be scaled up if needed for Rx Pharma products as well as VMS and cosmetic products as needed.

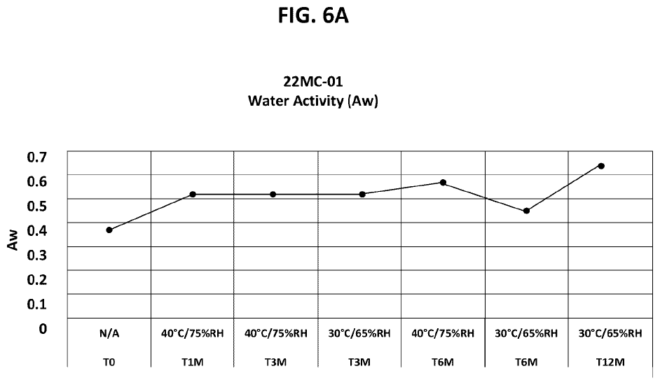

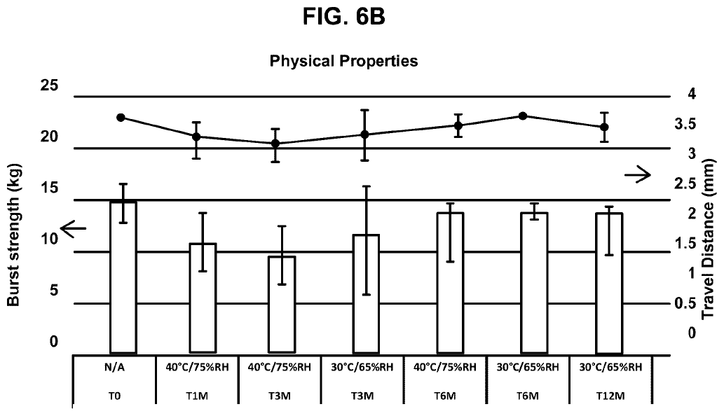

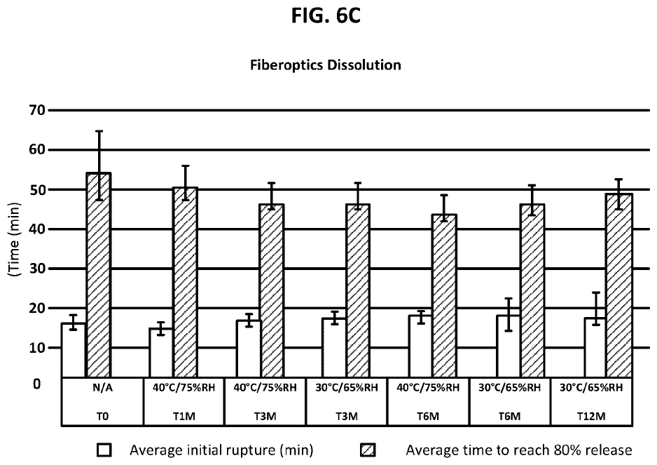

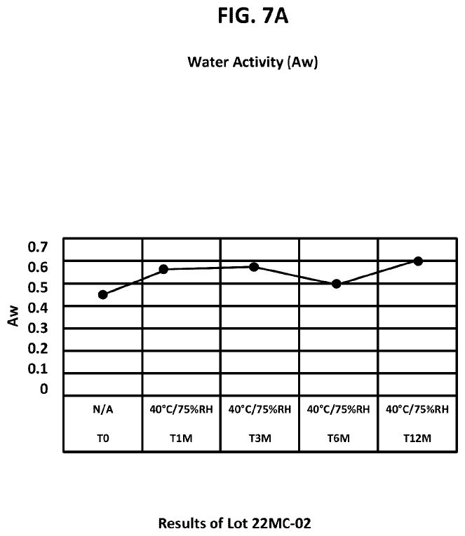

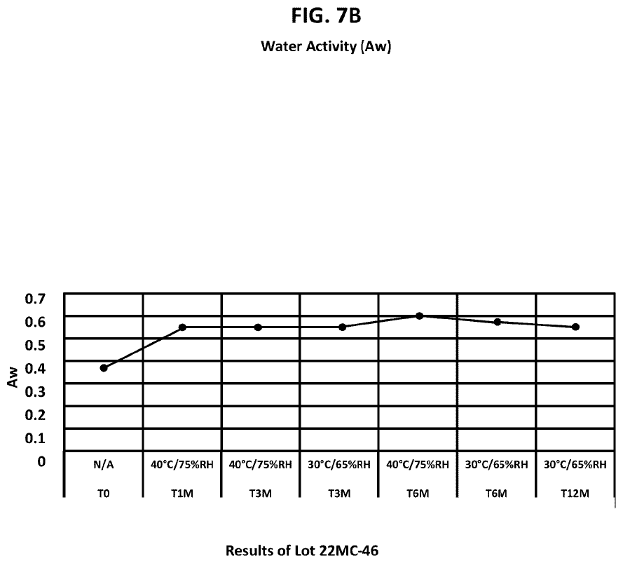

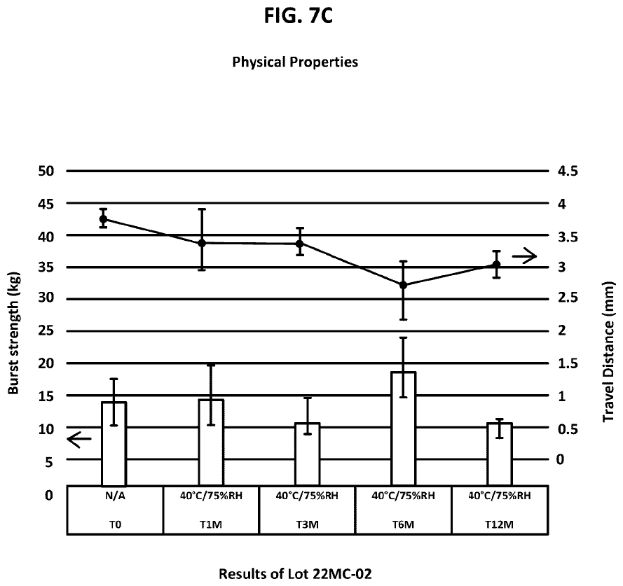

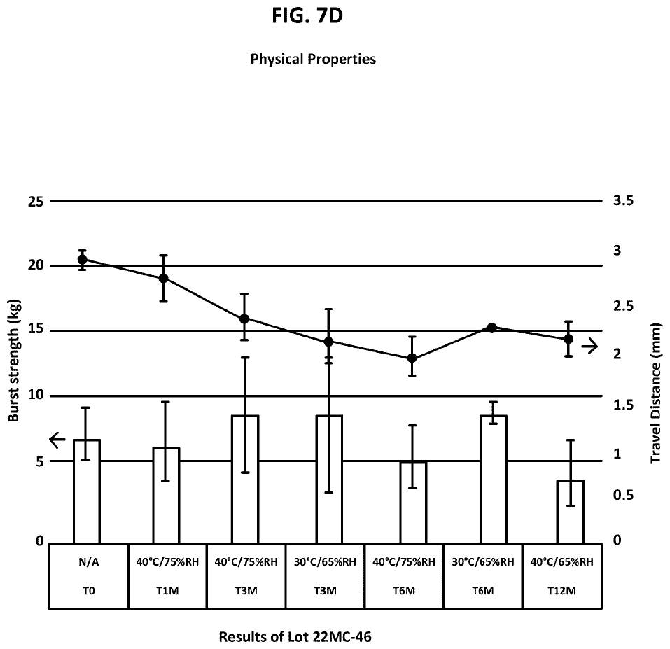

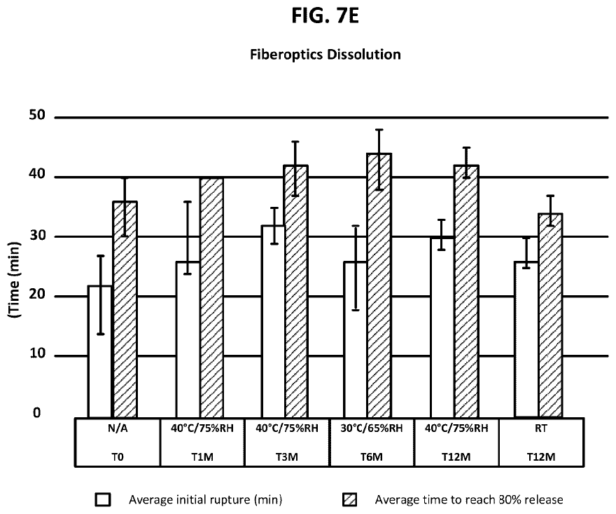

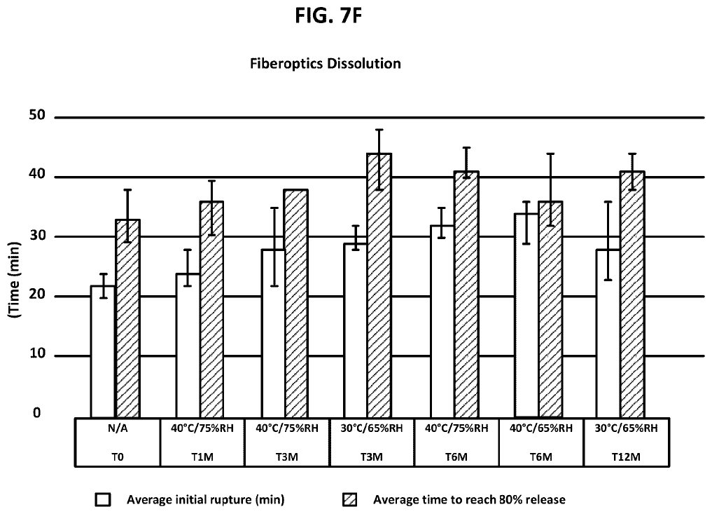

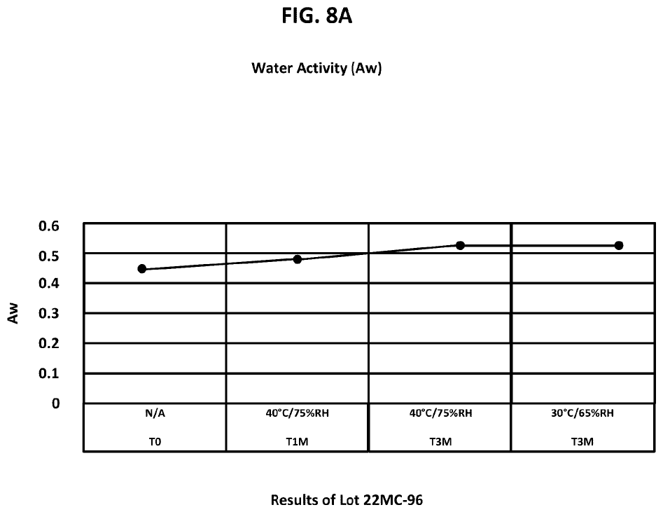

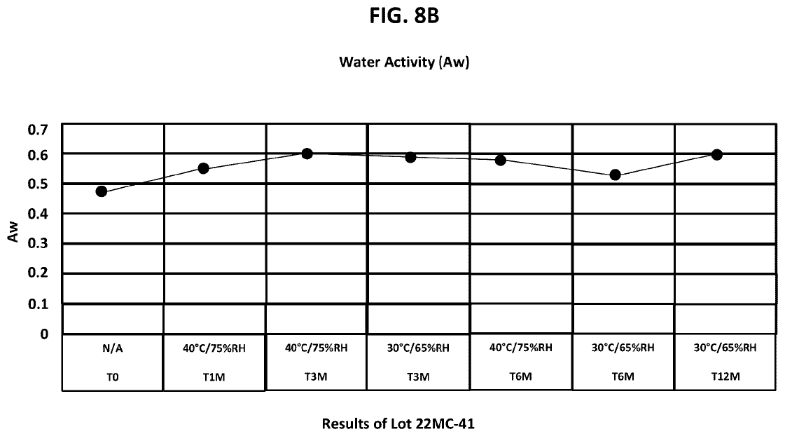

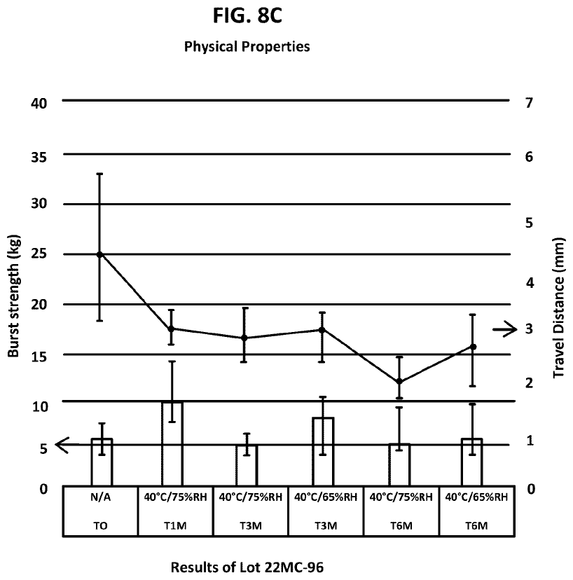

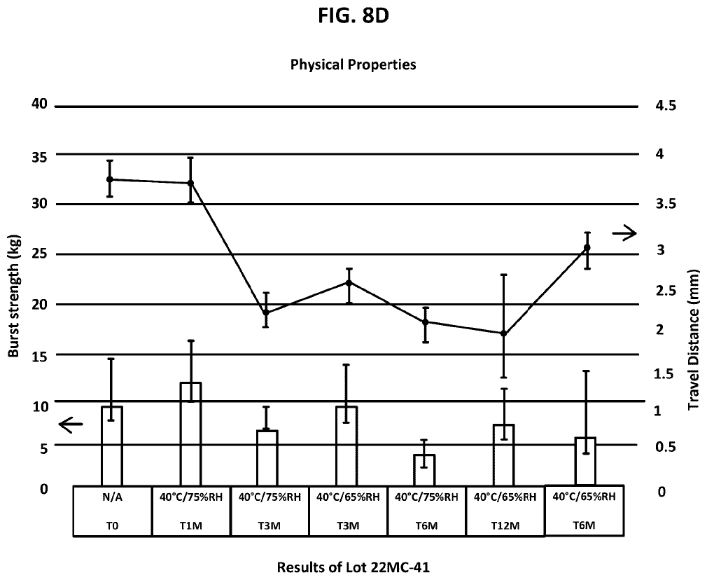

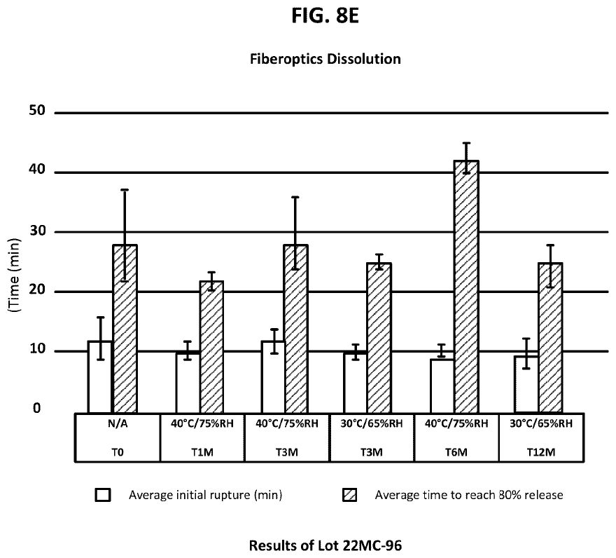

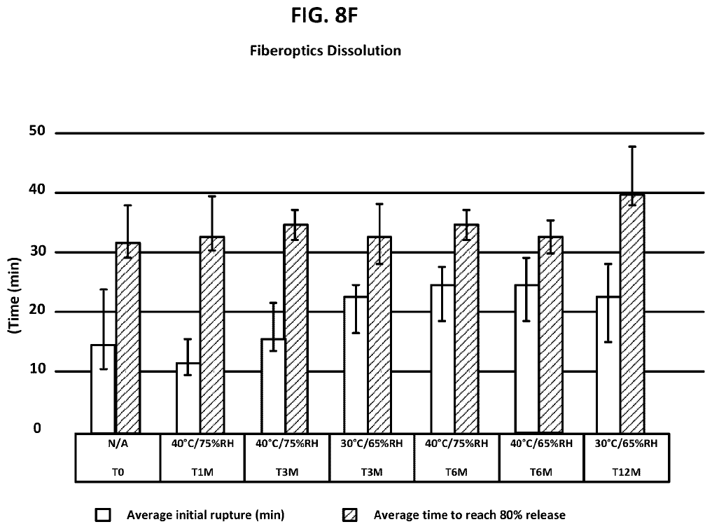

An informal stability study was also conducted for 12 months to determine the physical properties and dissolution performance after T 12 months. The informal stability study was initiated for 21MC-96, 22MC-01, 22MC-02, 22MC-41 and 22MC-46 capsules under 40° C./75% RH and 30° C./65% RH storage conditions. During this study, data was collected after T3M, T6M, and T12M. FIGS. 6A-6C represent the results of the stability study for lot 22MC-01. FIGS. 7A-7F show a comparison of the stability study for lot 22MC-02 and 22MC-46. FIGS. 8A-8F show a comparison of the stability study for lot 22MC-96 and 22MC-41. It was found that none of the softgel capsules in the study were detected to have leaked and the physical properties and dissolution performance was not affected significantly after being stored in accelerated conditions. Additionally, the results of this study were all within the error bar range and met the criteria for application in immediate release pharmaceutical dosage forms.

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail in order to avoid unnecessarily obscuring the present invention. Thus, the specific details set forth are exemplary. Particular embodiments may vary from these exemplary details and still be contemplated to be within the scope of the present invention.

Although the operations of the methods herein are described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

1. A softgel capsule comprising a fill material encapsulated by a film composition, wherein the film composition comprises a non-gelatin biobased polymer, wherein the film dissolves in less than 20 minutes according to a dissolution test using USP Apparatus paddle II at 75 rpm.

2. The softgel capsule of claim 1, wherein the non-gelatin biobased polymer comprises stearic acid, maltodextrin, pullulan, or a combination thereof.

3. The softgel capsule of claim 1, wherein the film further comprises a synthetic polymer.

4. The softgel capsule of claim 3, wherein the synthetic polymer is polyvinyl alcohol, polyvinyl alcohol-polyethylene glycol graft co-polymer, high molecular weight polyethylene glycol, povidone, a surfactant, a nonionic triblock copolymer or a combination thereof.

5. The softgel capsule of claim 4, wherein the synthetic polymer is a nonionic triblock copolymer.

6. The softgel capsule of claim 5, wherein the nonionic triblock copolymer comprises polyethylene oxide and polypropylene oxide blocks.

7. The softgel capsule of claim 4, wherein the surfactant is sodium lauryl sulfate.

8. The softgel capsule film of claim 1, wherein the film further comprises a non-animal derived gelling agent.

9. The softgel capsule film of claim 8, wherein the non-animal derived gelling agent comprises carrageenan, starch, pregelatinized starch, xanthan gum, agar, pectin, sugar, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, egg white powder, lacto albumin, soy protein, chitosan or a combination thereof.

10. The softgel capsule of claim 1, wherein the film further comprises a plasticizer.

11. The softgel capsule of claim 1, wherein the film further comprises a buffer agent.

12. The softgel capsule of claim 1, wherein the film dissolves in less than 15 minutes, less than 10 minutes, or less than 5 minutes.

13. The softgel capsule of claim 1, wherein the non-animal derived gelling agent comprises carrageenan, starch, or a combination thereof.

14. The softgel capsule of claim 1, wherein the ratio of carrageenan to starch is about 1:1 to about 1:10, about 1:1 to about 1:8, about 1:1 to about 1:5 or about 1:2.5 to about 1:4.5.

15-30. (canceled)

31. The softgel capsule of claim 2, wherein the polymer is stearic acid.

32. The softgel capsule of claim 31, wherein stearic acid is in an amount of about 0.5 to about 5% (w/w), about 1 to about 4% (w/w), or about 2 to about 3% (w/w) of the film.

33. The softgel capsule of claim 2, wherein the polymer is pullulan and stearic acid.

34. The softgel capsule of claim 33, wherein the polymer is in an amount of about 1.5 to about 25% (w/w), about 2 to about 20% (w/w), about 3 to about 18% (w/w), about 5 to about 15% (w/w), or about 7.5 to about 12.5% (w/w) of the film.

35-37. (canceled)

38. The softgel capsule of claim 1, wherein at least 80% of the fill material release within 30 minutes.

39. The softgel capsule of claim 1, wherein the fill material comprises an active agent.
